(19) United States
(12) Patent Application Publication
(10) Pub. No.: US 2008/0189838 A1
(43) Pub. Date: Aug. 14, 2008

(54) Multi-base lens goggle

(76) Inventor: Jerome J.M. Mage, Beverly Hills, CA (US)

Correspondence Address: STETINA BRUNDA GARRED & BRUCKER, 75 ENTERPRISE, SUITE 250, ALISO VIEJO, CA, 92656, US

(21) Appl. No.: 11/705,289

(22) Filed: Feb. 12, 2007

(51) Int. Cl. A61F 9/02

(52) U.S. Cl. 2/436

(57) ABSTRACT

A goggle is provided for protecting eyes of a wearer from projectiles and providing a wide field of view to the wearer through the goggle. In particular, the goggle may have a front portion and two side portions which are attached to lateral distal ends of the front portion. The two side portions may extend rearwardly to follow the contour of the wearer's head instead of the curvature of the front portion. Transitions between the front portion and the two side portions may be abrupt. Such configuration provides a compact goggle which permits the wearer to have a full range of motion required to maintain control and balance on a snowboard and a wide field of view through the goggle. The front portion may have a smaller base curvature compared to the base curvature of the two side portions.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The present invention relates to a goggle and more particularly, to a goggle permitting a wearer to have a field of view through the goggles about equal to the wearer's normal field of view.

Snowboarders traverse down a mountain by carving left to right. For regular stance riders, the rider leans toward the toe edge to turn right. As the rider turns right, the rider's body as well as the person's torso and head begins to align itself uphill to the mountain. To turn toward the left, the rider then turns his/her torso to the left and begins to apply pressure to the heel side edge of the board. At the end of the left turn, the rider's torso and head are now aligned downhill of the mountain. At the end of the left turn, the rider can see the entire downhill view of the mountain and can easily turn his/her torso toward the right and apply pressure to the toe side edge of the board to begin turning right again. The rider is able to see any obstacles (e.g., other snowboarders, skiers, poles, trees, etc.) downhill of the rider such that the rider can time his/her right turn maneuver to avoid colliding with the obstacles. In contrast, at the end of the right turn, the rider can see the entire uphill view of the mountain and the downhill view of the mountain is behind the rider. At this point, the rider is unable to see obstacles downhill of the rider. As such, the rider may not be able to see obstacles downhill of the rider and the rider may collide with the obstacle. To compensate for the lack of view downhill of the rider, the rider must keep track of other riders (i.e., dynamic obstacles, etc.) and poles (i.e., static obstacles, etc.) to avoid colliding with the obstacles. Further, the rider typically turns his/her torso and neck to gain a better view of the downhill side of the mountain to ascertain the position of the various obstacles downhill of the rider. Even further, the rider may shift his/her eyes toward the left to view obstacles downhill of the rider. Unfortunately, when the rider is wearing a goggle to prevent wind and snow from irritating the rider's eyes while traversing down the mountain, side members of the goggle block the rider's downhill view of the mountain and limits the rider's normal field of view in the lateral direction.

Accordingly, there is a need in the art for an improved goggle.

### BRIEF SUMMARY

The goggle discussed herein addresses the problems discussed above, discussed below and those that are known in the art.

The goggle protects the wearer's eyes from projectiles and provides a wide field of view through the goggle to the wearer. The goggle may comprise an injection molded frame having an upper member disposable adjacent to a forehead of the wearer, a lower member disposable adjacent to a nose and cheek of the wearer, and U shaped members having a base and two tines disposable adjacent to the wearer's temples. The two tines may be attached to the upper and lower members. The bases of the U shaped members may be laterally disposable within the wearer's near, mid or far peripheral vision or outside of the wearer's normal field of view for providing a wide field of view through the goggle to the wearer. For example, the bases may be laterally disposed within the wearer's near peripheral vision, mid-peripheral vision, or far peripheral vision. More preferably, the bases are laterally disposed outside of the wearer's normal field of view.

The upper member, lower member and two U shaped members may collectively form one lens receiving aperture across both eyes of the wearer. The lens receiving aperture may define an inner periphery.

The goggle may also comprise a first unitary multi-base lens having a front portion and two side portions integrally formed to lateral adjacent sides of the front portion. The front portion and two side portions may be formed from a single lens (e.g., injection molding process) or formed separately and subsequently attached to each other. A base curvature of the front portion of the lens may be smaller compared to base curvatures of the two side portions of the lens for forming a compact goggle. For example, the base curvature of the front portion may be five, whereas the base curvature of the side portions may be eight. Alternatively, the side portions may be flat.

The first unitary multi-base lens may define an outer periphery. The entire outer periphery of the first unitary multi-base lens may mate to the entire inner periphery of the single lens receiving aperture such that debris (e.g., snow, dirt, etc.) cannot pass into the goggle between the inner periphery of the goggle and the outer periphery of the lens for protecting the wearer's eyes from projectiles. Since the entire outer periphery of the first unitary multi-base lens mates with the entire inner periphery of the goggle, lateral distal ends of the two side portions extend within the wearer's near, mid or far peripheral vision or outside the wearer's normal field of view in the same manner that the bases are laterally disposable within the wearer's near, mid or far peripheral vision or outside the wearer's normal field of view. For example, lateral distal ends of the two side portions may extend within the wearer's near peripheral vision, mid-peripheral vision, and far peripheral vision. More preferably, the lateral distal ends of the two side portions are laterally disposed outside of the wearer's normal field of view.

The transitions between the front portion and two side portions may be abrupt. For example, the transitions between the front portion and two side portions may form a tight radius. By way of example and not limitation, the transition may have a radius of about ten (10) mm. Also, the transition may have a radius smaller than the curvature of the front portion and/or the side portions. Alternatively, the transition between the front portion and the two side portions may be smooth.

The lower member of the frame may have an air vent providing air communication between an outside, and an inside of the goggle for replacing air inside the goggle with air outside the goggle thereby moderating air temperature inside the goggle to approximate the air temperature outside the goggle. This moderates the air temperature inside of the goggle to approximate the temperature of a proximal surface of the first unitary multi-base lens to mitigate against fogging. Additionally, the air vent may have a cover which is selectively traversable over the air vent for closing the air vent.

The goggle may further have a second unitary multi base lens having a front portion and two side portions integrally formed to lateral adjacent sides of the front portion. The second unitary multi base lens may be received by the lens receiving aperture and offset behind or in front of the first unitary multi-base lens for providing an air gap to buffer the air temperature difference between the ambient temperature (i.e., air temperature outside the goggle) and a temperature of the air between the second unitary multi base lens and a face of the wearer (i.e., air-temperature inside the goggle).

A sealant may be disposed between outer peripheral portions of the first and second unitary multi base lenses for preventing moisture from entering a space between the first and second unitary multi base lenses. Removing moisture from the space between the first and second unitary multi base lenses also prevents moisture from forming on a proximal surface of the first unitary multi-base lens.

In another aspect of the goggle, the same may comprise an injection molded frame having an upper member disposable adjacent to a forehead of the wearer, a lower member disposable adjacent to a nose and cheek of the wearer, and U shaped members having a base and two tines disposable adjacent to the wearer's temples. The two tines may be attached to the upper and lower members. The upper member, lower member and two U shaped members may form one lens receiving aperture across both eyes of the wearer. The lens receiving aperture may define an inner periphery.

The goggle may also have a first unitary multi-base lens having a front portion and two side portions integrally formed to lateral adjacent sides of the front portions. A base curvature of the front portion may be smaller compared to base curvatures of the two side portions and the two side portions may extend rearward following a contour of the wearer's head instead of the base curvature of the front portion for forming a compact and contoured goggle. The first unitary multi-base lens may define an outer periphery. The entire outer periphery may mate to the entire inner periphery of the single lens receiving aperture extending the two side portions outside of the wearer's mid-peripheral vision for protecting the wearer's eyes from projectiles.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a top view of a goggle;

FIG. 2 is a front view of the goggle of FIG. 1;

FIG. 3 is a bottom view of the goggle of FIG. 2;

FIG. 4 is a side view of the goggle of FIG. 2;

FIG. 5 illustrates a wearer's normal field of view; and

FIG. 6 is a schematic comparing a prior art lens with a multi base lens.

### DETAILED DESCRIPTION

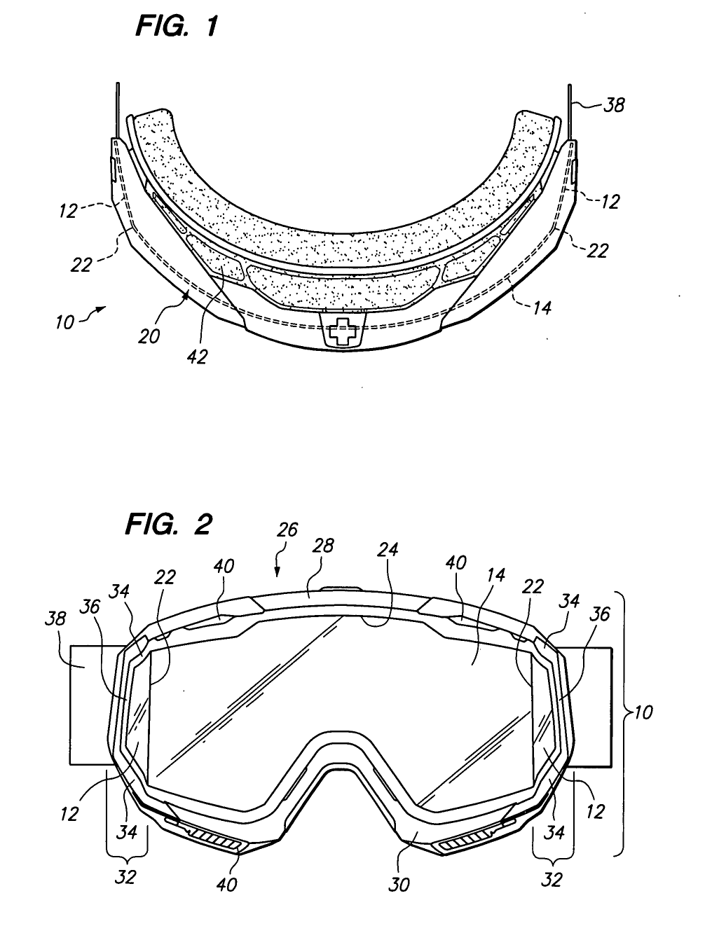

Referring now to the drawings which are for the purpose of illustration and not limitation, FIGS. 1-4 illustrates a snowboard goggle 10. The snowboard goggle 10 is used in snow conditions. The goggle 10 may be worn by a wearer in conditions below fifty (50) degrees Fahrenheit. Typically, a goggle 10 is worn during a snow storm or in other conditions where projectiles may enter the snowboarder's eyes thereby irritating the snowboarder's eyes and impairing his/her vision. Although the goggle 10 is discussed in relation to goggles for snowboarding, the teachings and goggle 10 disclosed herein are not limited to goggles for snowboarding. Rather, the teachings and goggle 10 discussed herein may be employed in goggles for other types of sports such as skiing and motocross (i.e., warmer climate sports) and the like.

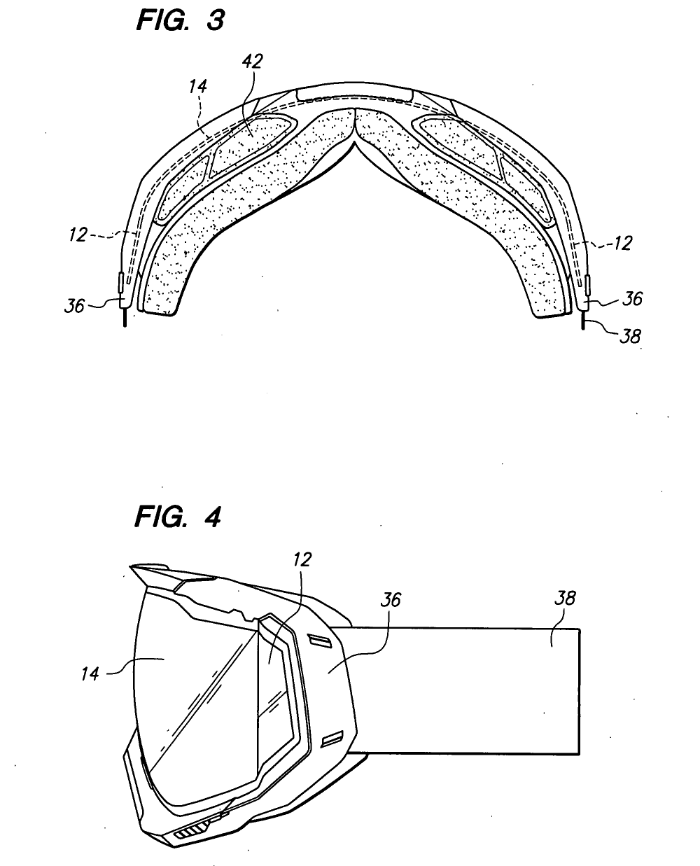

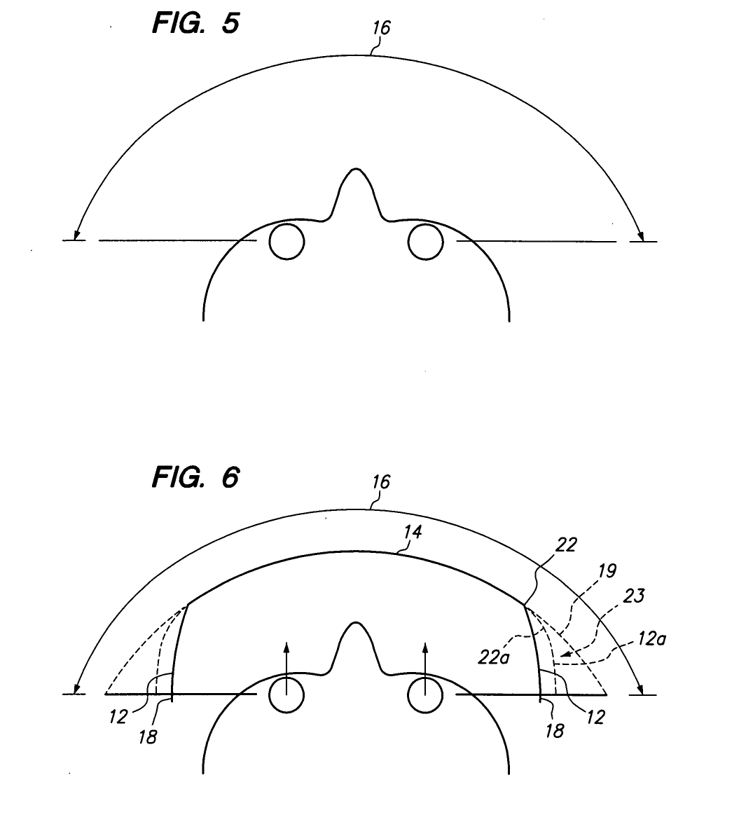

The goggle 10 shown in FIGS. 1-4 maximizes the wearer's field of view through the goggle 10. To this end, side portions 12 may be laterally and integrally formed to a front portion 14 in a rearward direction (see FIGS. 1 and 3) and adjacent the wearer's head such that as the wearer's eyes scan his/her surroundings from left to right, the wearer may be able to see objects to the left and right of the wearer. Also, distortion through the front portion 14 (see FIG. 1) may be reduced due to the low base curvature thereof in the lateral direction (see FIGS. 1 and 3). Even though the front portion 14 has a low base curvature in the lateral direction, the goggle 10 is ergonomically fitted to the wearer's head in that the front portion 14 does not extend outward and away from the wearer's head in an excessive manner. Rather, the two side portions 12 integrally formed to the front lens 14 follow the contour of the wearer's head instead of the base curvature of the front portion 14 in the lateral direction. In this manner, the goggle 10 permits the wearer to have a field of view through the goggle 10 about equal to a person's normal field of view 16. The normal field of view 16 is defined as a field of view of a person not wearing the goggle 10 while shifting his/her eyes left to right. Typically, the normal field of view 16 is about 180 degrees, as shown in FIG. 5. Also, the goggle 10 permits the wearer to have minimal distortion of his or her surrounding due to the low base curvature of the front portion 14.

In use, a snowboarder may wear the snowboard goggle 10 while traversing down a mountainside. As the snowboarder traverses down the mountainside, the snowboarder/wearer carves or turns left and right to control speed and to avoid obstacles (e.g., trees, poles, other snowboarders, other skiers, etc.). To link the left and right turns, the snowboarder must point his or her shoulders between about +/−90° with respect to a linear downward path of travel down the mountainside. At certain times, the snowboarder's back may be facing downhill of the mountainside. In this instance, the snowboarder's field of view is mainly directed uphill. However, the snowboarder's field of view should be directed downhill such that the snowboarder can see downhill obstacles to avoid. To compensate for the lack of downhill visibility, the snowboarder turns his or her head and torso to maintain visual contact downhill of the mountain. Unfortunately, people may not be sufficiently flexible to turn their torso and head to maintain visual contact with the downhill side of the mountain. To further maintain visual contact with the downhill side of the mountain, wearers may also shift their eyes. In prior art goggles, as discussed above, side members of the prior art goggles blocked the wearer's view even though the wearer shifts their eyes. Fortunately, the goggle 10 of the present invention has two clear side portions 12 which extend within the wearer's near, mid or far peripheral vision and more preferably outside of the wearer's normal field of view 16 (see FIG. 6) to permit the wearer to maintain visual contact with the downhill side of the mountain. Although FIG. 6 shows lateral distal ends 18 of the side portions 12 disposed outside of the wearer's normal field of view 16, it is also contemplated that the lateral distal ends 18 of the two side portions 12 may extend within the wearer's near peripheral vision, mid-peripheral vision or far peripheral vision. Preferably, the lateral distal ends 18 of the two side portions 12 extend outside of the wearer's normal field of view 16, as shown in FIG. 6. This permits the wearer to have a field of view through the goggle 10 equal to about the wearer's normal field of view 16. Accordingly, as the snowboarder turns his/her torso and neck and shifts his/her eyes, the wearer is better able to maintain visual contact with the downhill side of the mountain.

The front portion 14 of the goggle 10 may have a low base curvature in the lateral direction of the goggle 10 yet be ergonomically fitted to the wearer's head. In the prior art, if the front lens was fabricated with a low base curvature, the lateral distal ends of the front lens would extend outward and away from the wearer's head excessively. For example, if the front lens 14 were to provide 180 degree forward facing field of view through the goggle 10, then the front lens 14 must extend outward and away as shown by the dashed lines 19 in FIG. 6. When the lateral distal ends of the front lens 14 extends outward and away excessively, the goggle 10 becomes bulky and cumbersome. Unfortunately, a bulky goggle may interfere with the snowboarder's required head and torso movements to maintain control and balance down the mountainside. Also, the bulky goggle is aesthetically unappealing because of its space helmet look. Fortunately, in the present invention, two side portions 12 are integrally formed to lateral sides of the front portion 14 which extend rearwardly and follow the contour of the wearer's head instead of the base curvature or contour of the front portion 14. Hence, the goggle 10 is fitted to the wearer's head. The goggle 10 is compact and ergonomically fitted to the wearer's head.

As used herein, the normal field of view 16 is equal to a lateral range of sight of a person when the person shifts his/her eyes left to right. Typically, the person's normal lateral field of view is equal to or slightly greater than about 180°. The field of view through the goggles 10 is defined as a lateral range of sight that the wearer of the goggle 10 is able to see in the lateral direction while wearing the goggle 10. Typically, in prior art goggles, the field of view through the goggle is limited by side members of the frame of the goggle such that the field of view through prior art goggles is less than the normal field of view or may be less than the near-peripheral vision, mid-peripheral vision or the far peripheral vision.

As shown in FIG. 1, a first unitary multi-base lens 20 (shown by double dashed lines) may extend substantially across both eyes of the wearer. For example, the first portion may extend laterally across the eyes of the wearer such that the wearer is able to maintain visibility through the wearer's center of gaze when the person is looking straight ahead. Alternatively, the first portion may extend laterally across the eyes such that the wearer is able to maintain visibility through the wearer's center of gaze when the person shifts his/her eyes left to right.

The first unitary multi-base lens 20 which comprises the front portion 14 and the two side portions 12 may be fabricated from a unitary material (i.e., single lens) such that the front portion 14 and two side portions 12 are continuous from a left side to a right side. The base curvature of the front portion 14 in a vertical direction may be four. Also, the base curvature of the front lens 14 in a lateral direction may be five. Unfortunately, in the prior art, when the base curvature of the front lens in the lateral direction is five and the front lens extends laterally sufficient to permit the wearer to maintain visual contact with the mountain, as discussed in the background, lateral distal ends of the front lens extends excessively outward and beyond the wearer's face. As such, the goggle may be bulky and cumbersome so as to interfere with the required movements of the snowboarder to maintain control and balance while traversing down the mountain. Fortunately, in the present invention, distal lateral ends of the front portion 14 may be integrally formed to side portions 12 which extend rearwardly following the contour of the wearer's face side rather than the curvature of the front portion 14. This configuration produces a compact goggle 10 which provides less or no interference with the wearer's required movement to maintain control and balance while traversing down the mountain. Also, the side portions 12 permit the wearer to have a field of view through the goggle 10 equal to about the wearer's normal field of view 16, or the wearer's near, mid or far peripheral vision.

The front portion 14 and two side portions 12 may be fabricated as a unitary material. For example, the front portion 14 and the two side portions 12 may be injection molded from a clear plastic material. Alternatively, the front portion 14 and two side portions 12 may be separately formed and subsequently attached to each other. Also, the front portion 14 and the two side portions 12 may utilize ARC™ (Accurate Radius Curvature) polycarbonate lenses. These are shatterproof sport lenses which complement the natural curvature of the eye. The front portion 14 may be designed to become thinner as it moves away from the optical center, thus further eliminating distortion.

The front portion 14 and the side portions 12 may have different base curvatures. For example, the front portion 14 may have a base curvature of five in the lateral direction, whereas, the side portions 12 may have a base curvature of eight in the lateral direction. The transition 22 between the front portion 14 and the two side portions 12 may have a small radius. By way of example and not limitation, the transition 22 may have a radius of about ten (10) mm or less than ten (10) mm. Also, the transition 22 may have a radius smaller than the curvature of the front portion 14 and/or the side portions 12. The small radius of the transition 22 permits the goggle 10 to have a more compact configuration. For example, if a transition 22a had a wide radius, the two side lenses 12a would extend out more laterally than a transition 22 having a tight radius. Referring now to FIG. 6, the dashed line 23 illustrates the transition 22a having a wide radius. As shown, the two side lens 12a joined to the front lens 14 extends out more laterally compared to the two side lens 12 joined to the front lens 14 with a transition 22 having a tight radius.

The lens may also be treated with various coatings, such as anti-scratch hardcoats, to make the lenses scratch resistant. Furthermore, the lenses may be treated with an anti-fog coating to help inhibit the formation of fog on the lens. The lens may also be ARC™ Polarized Lenses which are designed to diffuse blinding glare that bounces off of flat surfaces such as water and pavement. ARC™ Polarized Lens accomplishes this by utilizing an advanced polarization filter which is bonded between two layers of ARC™ polycarbonate which are used to form each lens.

The lens may also utilize CR-39 technology which offers unique colorations and/or lens tint options, along with being lightweight while being compatible with acetate frames 26. For example, the lens tint options may include, but is not limited to the following: gray which is good for general-purpose lenses which offer true color perception; bronze which sharpens contrast and increases depth perception; clear which is used for low light conditions; green-gray which is considered a good general purposes lens; high-intensity yellow which brightens low level light conditions; orange which sharpens contrast and is used in flat light conditions; bronze with gold which is the same tint as bronze and includes a gold coating; gray with silver which is the same as gray and also includes a silver coating; bronze with silver which is the same as bronze and also includes a silver coating; and gray polarized or bronze polarized which diffuse blinding glare that bounces off of flat surfaces such as water and pavement.

Although the goggle 10 is discussed in relation to a single unitary lens which is disposed across the wearer's face. It is also contemplated that the goggle 10 may be a dual lens goggle 10. In particular, the front portion 14 and two side portions 12 fabricated in accordance with the description given above may be referred to as a first unitary multi-base lens 20. The first unitary multi-base lens 20 may be gapped away or offset from a second unitary multi base lens which matches the general contour of the first unitary multi-base lens 20. For example, the second unitary multi base lens may be offset or gapped away from the first unitary multi-base lens 20 about 3 mm. A sealant (e.g., polyurethane based glue, etc.) may be interposed between the first and second unitary multi base lenses about outer peripheries of the first and second unitary multi base lenses. The sealant provides an airtight and moisture tight seal between the first and second unitary multi base lenses. The sealant and the first and second lenses may define an inner cavity. Moisture from the inner cavity may be evacuated from the inner cavity to prevent formation of fog on a proximal surface of the first lens and a distal surface of the second lens. The dual lenses mitigate against fogging by buffering a temperature differential between a distal surface of the first lens and a proximal surface of the second lens via the inner cavity. In particular, the temperature differential between the distal surface of the first lens and the proximal surface of the second lens is split between the temperature difference between the distal and proximal surfaces of the first lens and the distal and proximal surfaces of the second lens. Since the temperature difference between the distal and proximal surfaces of the first and second lens are reduced, fog is less likely to form on the surfaces of the first and second lenses.

The first unitary multi-base lens 20 may be received into a lens receiving aperture 24 of a frame 26 of the goggle 10. The frame 26 may have an upper member 28 (see FIG. 2) which extends across a forehead of the wearer. The frame 26 may also have a lower member 30 which extends across cheeks of the wearer and is sized and configured to fit over the nose of the wearer. The upper and lower members 28, 30 may be connected to each other via side members 32. Each of the side members 32 may define two tines 34 and a base 36. The two tines 34 may be attached to the upper and lower members 28, 30. The base 36 may extend rearwardly to about the maximum range of the wearer's normal field of view 16. An inner periphery of the frame 26 defined by the upper member 28, lower member 30 and two side members 32 may have a first groove which is sized and configured to receive an outer periphery of the first unitary multi-base lens 20. Such groove retains or attaches the first unitary multi-base lens 20 to the frame 26. It is also contemplated that the inner periphery of the frame 26 may have two grooves (i.e., first and second grooves) offset from each other. The first groove may receive the first lens and the second groove may receive the second lens to gap the second lens away from the first lens.

A strap 38 may be attached to the goggle 10 for holding the goggle 10 to the wearer's head. For example, the strap 38 may be attached to the bases 36 of the side members 32. The frame 26 may be mounted onto the wearer's face and the strap 38 may extend around a backside of the wearer's head to hold the goggle 10 on the wearer's face.

The goggle 10 may have a plurality of vents which permit air to flow into and out of the goggle 10 or between an inside and an outside of the goggle 10. The inside of the goggle 10 is defined as air space between the first or second lenses and the wearer's face. The outside of the goggle 10 is defined as air space in front of the first lens and frame 26 of the goggle 10. A front side of the upper and lower members 28, 30 may have vent holes 40 formed therethrough which permit air outside of the goggle 10 to flow into the inside of the goggle 10. A topside (see FIG. 1) of the upper member 28 and a bottom side (see FIG. 3) of the lower member 30 may also have vent holes 42 which permit air inside of the goggle 10 to escape or flow to the outside of the goggle 10. In this manner, the air that enters the vent holes 40 in the front surfaces of upper and lower members 28, 30 displaces the air inside of the goggle 10s through the vent holes 42 formed on the top and bottom surfaces of the upper and lower members 28, 30, respectively. The vent holes 42 formed in the top and bottom surfaces of the upper and lower members 28, 30 may be larger compared to the vent holes 40 formed in the front surfaces of the upper and lower members 28, 30 to prevent any back pressure such that air may freely flow into the inside of the goggle 10 and displace air inside of the goggle 10 to the outside of the goggle 10 through the vent holes 42 formed in the top and bottom surfaces of the upper and lower members 28, 30.

At least one of the vent holes 40 formed in the front surfaces of the upper and lower members 28, 30 may have a cover which is slideably traversable over the vent hole 40 to selectively open and close the vent hole.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A goggle for protecting eyes of a wearer from projectiles and providing a wide field of view through the goggle to the wearer, the goggle comprising:
a) an injection molded frame having an upper member disposable adjacent to a forehead of the wearer, a lower member disposable adjacent to a nose and cheek of the wearer, U shaped members having a base and two tines disposable adjacent to the wearer's temples, the two tines being attached to the upper and lower members, the upper member, lower member and two U shaped members forming one lens receiving aperture across both eyes of the wearer, the lens receiving aperture defining an inner periphery; and
b) a first unitary multi-base lens having a front portion and two side portions attached to lateral adjacent sides of the front portion, a base curvature of the front portion being smaller compared to base curvatures of the two side portions and the two side portions following a head contour of the wearer more than a base curvature of the front portion for forming a compact goggle, transitions between the front portion and the two side portions being abrupt, the first unitary multi-base lens defining an outer periphery, the entire outer periphery being mated to the entire inner periphery of the single lens receiving aperture for protecting the wearer's eyes from projectiles.

2. The goggle of claim 1 wherein the two side portions extend rearwardly for fitting the goggle to the wearer's head.

3. The goggle of claim 1 wherein the abrupt transitions have a configuration of a tight radius.

4. The goggle of claim 1 wherein the base curvature of the front portion is about five and the base curvature of each of the side portions is about eight.

5. The goggle of claim 1 wherein the lower member has an air vent providing air communication between an outside and an inside of the goggle for replacing air inside the goggle with air outside the goggle thereby moderating air temperature inside the goggle to mitigate against fogging.

6. The goggle of claim 5 wherein a cover is selectively traversable over the air vent for closing the air vent.

7. The goggle of claim 1 further comprising a second unitary multi base lens having a front portion and two side portions attached to lateral adjacent sides of the front portion, the second unitary multi base lens received by the lens receiving aperture and offset from the first single piece multi base lens for providing an air gap to buffer a temperature difference between the ambient temperature and a temperature of the air between the second unitary multi base lens and a face of the wearer.

8. The goggle of claim 7 further comprising a sealant disposed between outer peripheral portions of the first and second unitary multi base lenses for preventing moisture from entering a space between the first and second unitary multi base lenses.

9. The goggle of claim 1 wherein the two side portions are flat.

10. A goggle for protecting eyes of a wearer from projectiles and providing a wide field of view to the wearer through the goggle, the goggle comprising:
a) an injection molded frame having an upper member disposable adjacent to a forehead of the wearer, a lower member disposable adjacent to a nose and cheek of the wearer, U shaped members having a base and two tines disposable adjacent to the wearer's temples, the two tines being attached to the upper and lower members, the bases of the U shaped members being laterally disposed to at least the wearer's mid peripheral vision for providing the wide field of view to the wearer through the goggle, the upper member, lower member and two U shaped members forming one lens receiving aperture across both eyes of the wearer, the lens receiving aperture defining an inner periphery; and
b) a first unitary multi-base lens having a front portion and two side portions attached to lateral adjacent sides of the front portion, a base curvature of the front portion being smaller compared to base curvatures of the two side portions for forming a compact goggle, the first unitary multi-base lens defining an outer periphery, the entire outer periphery being mated to the entire inner periphery of the single lens receiving aperture extending the two side portions to at least the wearer's mid peripheral vision for protecting the wearer's eyes from projectiles.

11. The goggle of claim 10 wherein the bases of the U shaped members are laterally disposed to at least the wearer's far peripheral vision, and the two side portions are extended to at least the wearer's far peripheral vision.

12. The goggle of claim 10 wherein the bases of the U shaped members are laterally disposed outside of the wearer's normal field of view, and the two side portions are extended outside of the wearer's normal field of view.
